(19) United States
(12) Patent Application Publication
(10) Pub. No.: US 2022/0361945 A1
(43) Pub. Date: Nov. 17, 2022

(54) Methods And Apparatus For Heart Valve Leaflet Modification

(71) Applicant: AMX Technologies, LLC, Plymouth, MN (US)

(72) Inventors: Paul Sorajja, Minneapolis, MN (US); Daniel Coyle, Saint Louis Park, MN (US); Richard Farrell, White Bear Lake, MN (US); John Matthew Swoyer, Blaine, MN (US)

(73) Assignee: AMX Technologies, LLC, Plymouth, MN (US)

(21) Appl. No.: 17/755,614

(22) PCT Filed: Nov. 9, 2020
(86) PCT No.: PCT/US2020/059719

§ 371 Date: May 3, 2022

Related U.S. Application Data
(60) Provisional application No. 62/933,007, filed on Nov. 8, 2019.

(51) Int. Cl. A61B 18/14

(52) U.S. Cl. CPC A61B 18/1492; A61B 2018/00369

(57) ABSTRACT

Systems, devices and methods for percutaneously modifying leaflets within the heart, thereby facilitating further repair or replacement. In some embodiments, the leaflets are cut. In other embodiments, the leaflets are removed either in part or in whole. The modifications to the leaflets may be made in conjunction with a prosthetic valve or independently.

### RELATED APPLICATIONS

This application claims priority to U.S. Provisional Application Ser. No. 62/933,007 filed Nov. 8, 2019 entitled Heart Valve Leaflet Modification, which is hereby incorporated herein by reference in its entirety.

### FIELD OF THE INVENTION

The present disclosure relates to novel and advantageous transcatheter-delivered valve repair devices. More specifically, the devices herein address issues related to treatment of pathology involving the heart valves, such as the mitral, aortic, pulmonary, and tricuspid valves.

### BACKGROUND OF THE INVENTION

The background description provided herein is for the purpose of generally presenting the context of the disclosure. Work of the presently named inventor(s), to the extent it is described in this background section, as well as aspects of the description that may not otherwise qualify as prior art at the time of filing, are neither expressly nor impliedly admitted as prior art against the present disclosure.

One common cardiovascular pathology is valvular heart disease, which may consist of narrowing (i.e., stenosis), incompetence (i.e., insufficiency or regurgitation), or a combination of these two diseases affecting the heart valves in a patient. When valvular heart disease occurs, the chambers of the heart can adversely remodel, leading to heart failure, severe morbidity, and impaired survival.

Open surgery is commonly performed but many patients are not candidates due to procedural risk. In many instances, a minimally invasive approach for treatment is preferred to minimize pen-operative and post-operative recovery.

Transcatheter approaches to treat valvular heart disease involve repairing the valve or performing a complete replacement, whereby a prosthesis is implanted inside the patient's native valve. In other cases, a prosthesis may be implanted within a previously placed prosthesis that has become dysfunctional or dislodged (e.g., valve-in-valve).

Transcatheter therapies for valvular heart disease commonly preserve the native leaflets or prosthetic material. Unfortunately, the native leaflets or prosthetic material that is left in place can interfere with the success of subsequent repair or replacement. In some instances, the risk of interference is too high and successful transcatheter therapy is not possible. In other instances, the interference is not noticed until after the valve has been implanted.

One specific example is transcatheter mitral valve replacement, in which a valvular prosthesis is implanted in either a patient's native mitral valve apparatus or a previously placed prosthesis. Due to the proximity of the mitral valve annulus to the left ventricular outflow tract, transcatheter mitral valve replacement may lead to positioning of the native or prosthetic leaflets in the direction of systolic flow. Severe left ventricular outflow tract obstruction may result from anterior positioning of the native leaflet or prosthetic material and can be life-threatening.

### OBJECTS AND SUMMARY OF THE INVENTION

The following presents a simplified summary of one or more embodiments of the present disclosure in order to provide a basic understanding of such embodiments. This summary is not an extensive overview of all contemplated embodiments and is intended to neither identify key or critical elements of all embodiments, nor delineate the scope of any or all embodiments.

The present disclosure relates to systems and methods for percutaneously modifying leaflets within the heart, thereby facilitating further repair of replacement. In some embodiments, the leaflets are cut. In other embodiments, the leaflets are removed either in part or in whole. The disclosure consists of a steerable guide catheter (SGC) that is placed into the cardiac chamber, a delivery catheter (DC), a cutting element (CU), a piercing member (P) with barbs or retention elements (B). The disclosure describes methods for modifying leaflets in the mitral valve, but these same methods and tools can be used for any leaflet within the heart, either preceding subsequent valve therapy (i.e., repair or replacement), or as standalone therapy.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

One aspect of the invention is a valve leaflet modification device comprising: a steerable guide catheter; a piercing element extendable from a distal end of the steerable guide catheter and having tissue engagement features; a cutting element extendable from the distal end of the steerable guide catheter and expandable from a first configuration to a second configuration; wherein said cutting element and said piercing element are translatable relative to each other to trap a valve leaflet between the cutting element and the piercing element, allowing the cutting element to modify the leaflet.

Another aspect of the invention is a system for making modifications to native leaflets comprising: a steerable guide catheter; a delivery catheter extendable from a distal end of the steerable guide catheter; a cutting element extendable from the distal end of the steerable guide catheter and expandable from a first configuration to a second configuration, the cutting element including: a cutting unit having a distal crossing portion; a push rod; and, a connecting element that connects the cutting unit to the push rod.

Another aspect of the invention is a method for treating a valve leaflet comprising: advancing a cutting element through a catheter to a targeted valve leaflet; expanding the cutting element from a first configuration to a second configuration; engaging the valve leaflet with the cutting element; and, modifying the valve leaflet with the cutting element.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which:

FIG. 1 is a diagram of the human heart provided as a reference;

FIG. 2 is a diagram of a human heart with a prosthetic mitral valve installed therein provided to show the problem to which the present invention is directed;

FIG. 3A is a perspective view of an unmodified mitral valve provided as a reference;

FIG. 3B is a perspective view of a mitral valve having been modified according to an embodiment of the invention;

FIG. 4A is a plan view of a mitral valve having been modified according to an embodiment of the invention;

FIG. 4B is a plan view of a mitral valve having been modified according to an embodiment of the invention;

FIG. 5A is a diagram of a step of an embodiment of a leaflet modification method of the invention;

FIG. 5B is a diagram of a step of an embodiment of a leaflet modification method of the invention;

FIG. 5C is a diagram of a step of an embodiment of a leaflet modification method of the invention;

FIG. 5D is a diagram of a step of an embodiment of a leaflet modification method of the invention;

FIG. 5E is a diagram of a step of an embodiment of a leaflet modification method of the invention;

FIG. 5F is a diagram of a step of an embodiment of a leaflet modification method of the invention;

FIG. 5G is a diagram of a step of an embodiment of a leaflet modification method of the invention;

FIG. 6A is a perspective view of a mitral valve having been modified according to an embodiment of the invention;

FIG. 6B is a perspective view of a mitral valve having been modified according to an embodiment of the invention;

FIG. 6C is a perspective view of a mitral valve having been modified according to an embodiment of the invention;

FIG. 6D is a perspective view of a mitral valve having been modified according to an embodiment of the invention;

FIG. 6E is a perspective view of a mitral valve having been modified according to an embodiment of the invention;

FIG. 6F is a perspective view of a mitral valve having been modified according to an embodiment of the invention;

FIG. 7A is a diagram of a step of an embodiment of a leaflet modification method of the invention;

FIG. 7B is a diagram of a step of an embodiment of a leaflet modification method of the invention;

FIG. 7C is a diagram of a step of an embodiment of a leaflet modification method of the invention;

FIG. 7D is a diagram of a step of an embodiment of a leaflet modification method of the invention;

FIG. 7E is a diagram of a step of an embodiment of a leaflet modification method of the invention;

FIG. 7F is a diagram of a step of an embodiment of a leaflet modification method of the invention;

FIG. 7G is a diagram of a step of an embodiment of a leaflet modification method of the invention;

FIG. 7H is a diagram of a step of an embodiment of a leaflet modification method of the invention;

FIG. 8 is a plan view of a modified aortic valve having been modified by an embodiment of a leaflet modification method of the invention;

FIG. 9A is a diagram of a step of an embodiment of a leaflet modification method of the invention;

FIG. 9B is a diagram of a step of an embodiment of a leaflet modification method of the invention;

FIG. 10A is a plan view of a modified aortic valve having been modified by an embodiment of a leaflet modification method of the invention;

FIG. 10B is a plan view of a modified aortic valve having been modified by an embodiment of a leaflet modification method of the invention;

FIG. 10C is a plan view of a modified aortic valve having been modified by an embodiment of a leaflet modification method of the invention;

FIG. 10D is a plan view of a modified aortic valve having been modified by an embodiment of a leaflet modification method of the invention;

FIG. 10E is a plan view of a modified aortic valve having been modified by an embodiment of a leaflet modification method of the invention;

FIG. 11 is a side elevation of an embodiment of a leaflet modification device of the invention;

FIG. 12 is a side elevation of an embodiment of a leaflet modification device of the invention;

FIG. 13 is a side elevation of an embodiment of a leaflet modification device of the invention;

FIG. 14 is a side elevation of an embodiment of a leaflet modification device of the invention;

FIG. 15 is a side elevation of an embodiment of a leaflet modification device of the invention;

FIG. 16 is a side elevation of an embodiment of a leaflet modification device of the invention; and,

FIG. 17 is a side elevation of an embodiment of a leaflet modification device of the invention.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

The present invention relates to treatment of pathology in the aortic root and ascending aorta using devices deployed via a catheter. Although the present disclosure discusses the embodiments herein with respect to a patient's aortic root and ascending aorta, the embodiments are applicable to any valve of the patient's heart and the disclosure herein must not be construed as to being limited to this application. The embodiments described herein may be applicable to repair of other valves and chambers of the human heart.

When used in the patient, the device of the present invention modifies the leaflets to reduce risk of Left Ventricular Outflow Tract (“LVOT”) obstruction and to facilitate subsequent repair or replacement of the valve. This device can be steered to any portion of any valve leaflet, and either cut or remove the targeted portions, either in part or in whole. From the perspective of the operator, the device implantation may be guided by echocardiography and fluoroscopy with real time assessment. The cutting element can be different shapes chosen to suit the patient's anatomy and desire for facilitating subsequent valvular therapy.

A heart commonly consists of four valves, the mitral, aortic, pulmonic, and tricuspid valves. For description purposes, the present disclosure focuses on use of the present device and methods for modifying cardiac leaflets.

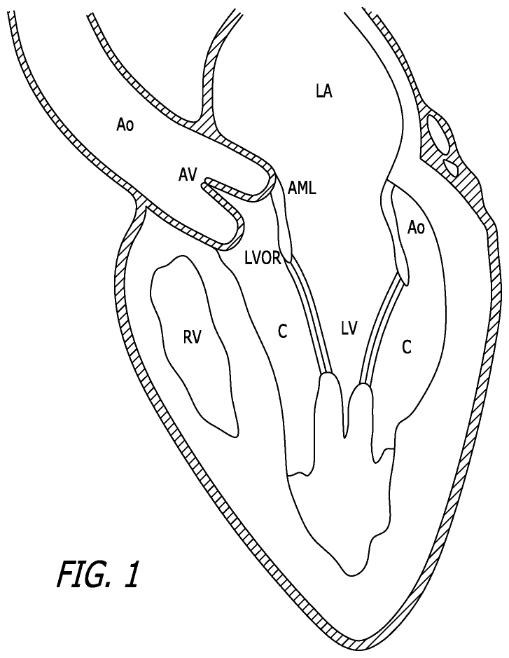

FIG. 1 shows the various parts of the human heart, provided by way of reference and establishing some of the acronyms used herein. The anterior mitral leaflet AML is part of the cardiac skeleton, near the aortic valve AV, and close to the left ventricular outflow tract LVOT. The proximity of the AML to the LVOT increases in diastole and decreases in systole, when systolic pressures lead to mitral valve closure. Thus, systolic flow through the LVOT is typically not impeded by the AML.

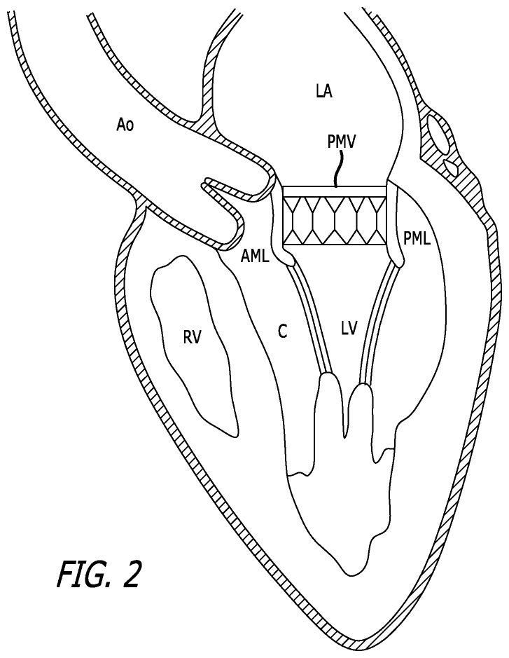

As shown in FIG. 2, when mitral valve replacement occurs by replacing the mitral valve with a prosthetic mitral valve PMV, the AML typically becomes fixed external to the frame of the prosthesis. This fixation of the AML then creates impedance to systolic flow through the LVOT, and such obstruction can be life-threatening. In many instances, mitral valve replacement, especially using transcatheter means, is not performed due to the LVOT obstruction from AML fixation.

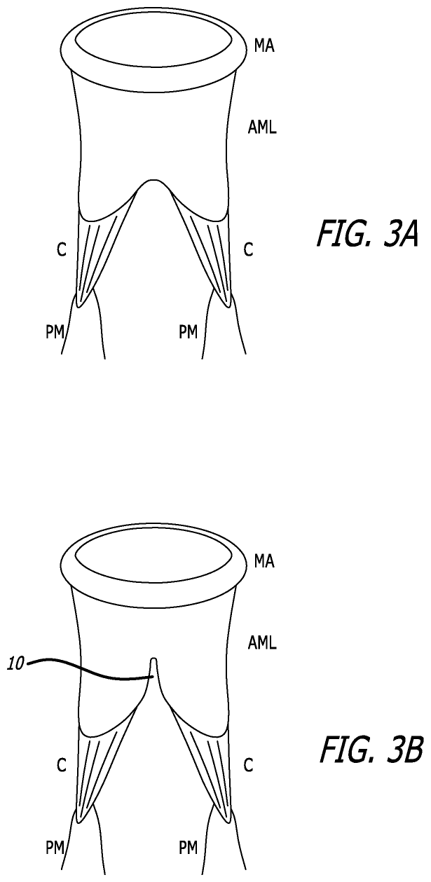

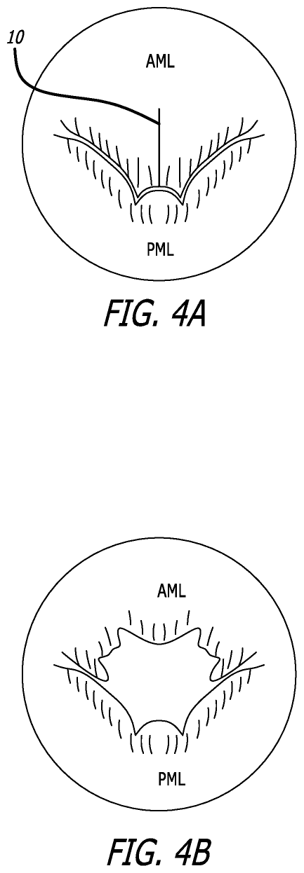

When a patient undergoes open surgery, the surgeon may excise the AML to facilitate mitral valve therapy. The present invention is directed to device and methods usable to modify the AML and thereby reduce the risk of LVOT obstruction. This modification changes the AML from its baseline state (FIG. 3A) to a configuration (FIG. 3B) having an embodiment of a modification 10 where the anterior portion and its free edges allow blood flow through the cells of the valve prosthesis that is subsequently placed. In one non-limiting example of an embodiment shown in FIG. 3B, the modification 10 is only a cut that preserves leaflet coaptation during systolic closure of the mitral valve. FIG. 4A is a plan view of a MV showing an example of a position of the modification 10 during the coaptation of the anterior mitral leaflet AML with the posterior mitral leaflet PML. In another embodiment, the modification is partial or complete removal of the leaflet (FIG. 4B), which would terminate the use of the native mitral valve in favor of the implantation of a prosthetic mitral valve.

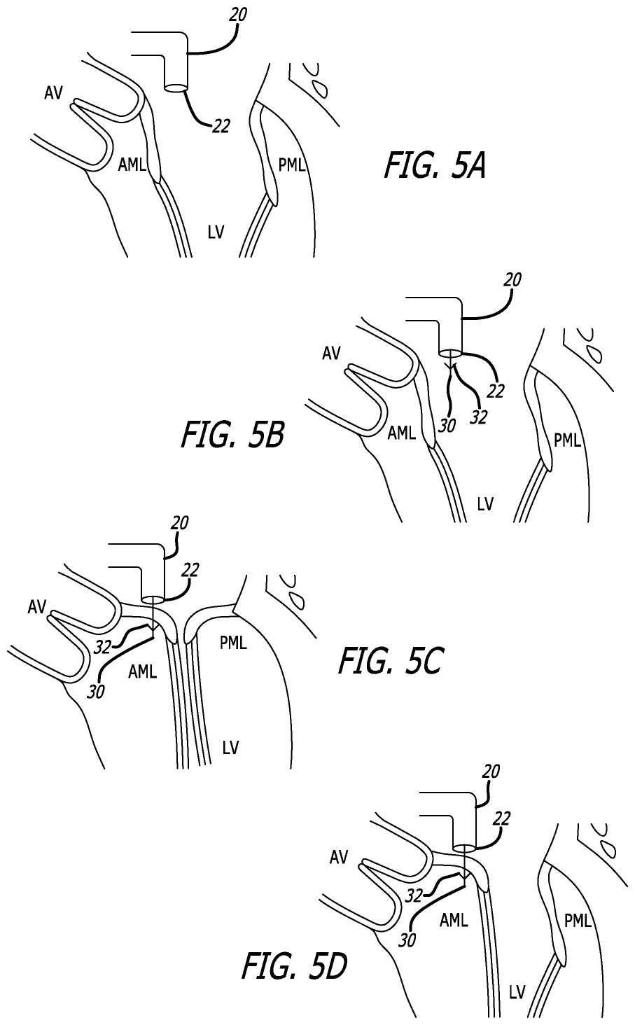

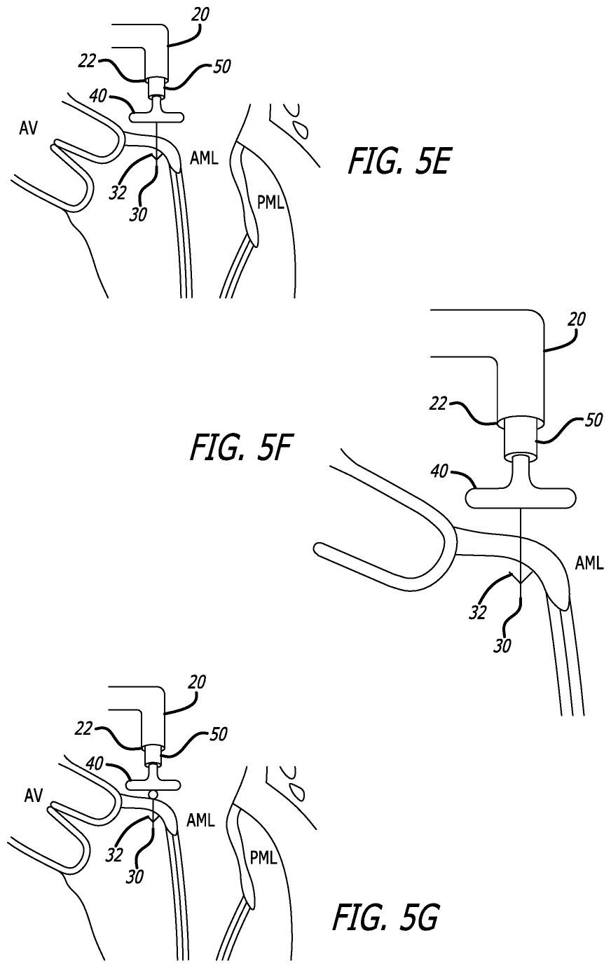

Referring to FIGS. 5A-5G, an embodiment of a device and method of the invention is described. Beginning with FIG. 5A, a steerable guide catheter 20 is navigated to the left atrium and oriented such that a distal end 22 of the catheter 20 is directed toward the anterior mitral leaflet AML. One skilled in the art will understand that this method may be used to modify other anatomical valve and that this method is provided by way of nonlimiting example.

Next, as seen in FIG. 5B, a piercing element 30 is advanced out of the distal end 22 of the steerable guide catheter 20. The piercing element 30 includes tissue engagement features 32, such as barbs or similar retention elements, leading to fixation of the AML in both systole (FIG. 5C), in which the AML and PML coapt, and diastole (FIG. 5D), in which the PML is directed away from the AML by the flow of blood from the left atrium LA to the left ventricle LV. The piercing element 30 may include a hypotube (not shown) for placement of a guidewire through the AML.

Referring to FIGS. 5E and 5F, once the AML is fixed, a cutting element 40 is advanced through a delivery catheter 50, which is translatably contained within the steerable guide catheter 20, to engage the AML for modification. Piercing methodologies that can be used include for example, RF energy, mechanical force of the leaflets closing and hitting a needle point. Various embodiments of the cutting element 40 are described in more detail below.

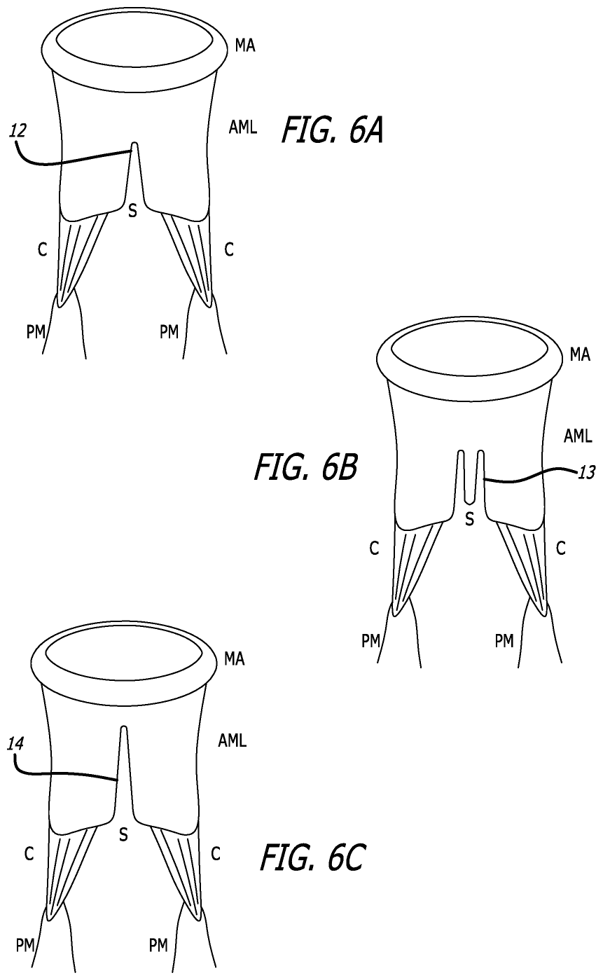

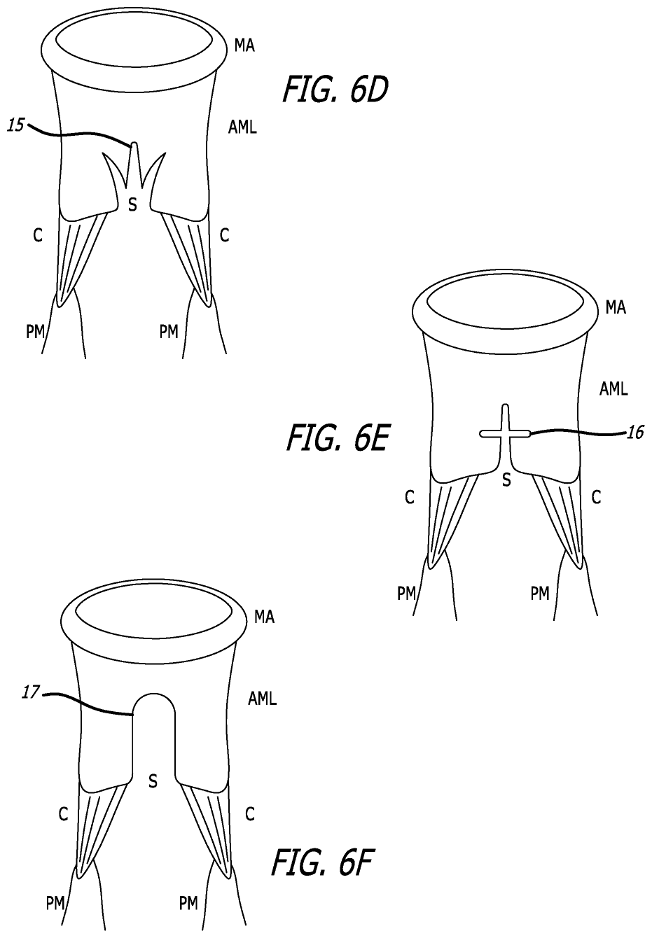

FIGS. 6A-6F show different non-limiting examples of modifications that may be made to the AML (or other leaflets) according to the invention. In FIG. 6A, the modification 10 is embodied as a single slit 12. FIG. 6B shows a double slit 13. FIG. 6C shows an elongated slit 14. FIG. 6D shows a crown-shaped slit 15. FIG. 6E shows a cross-shaped slit 16. FIG. 6F shows a wide slit 17 involving tissue resection.

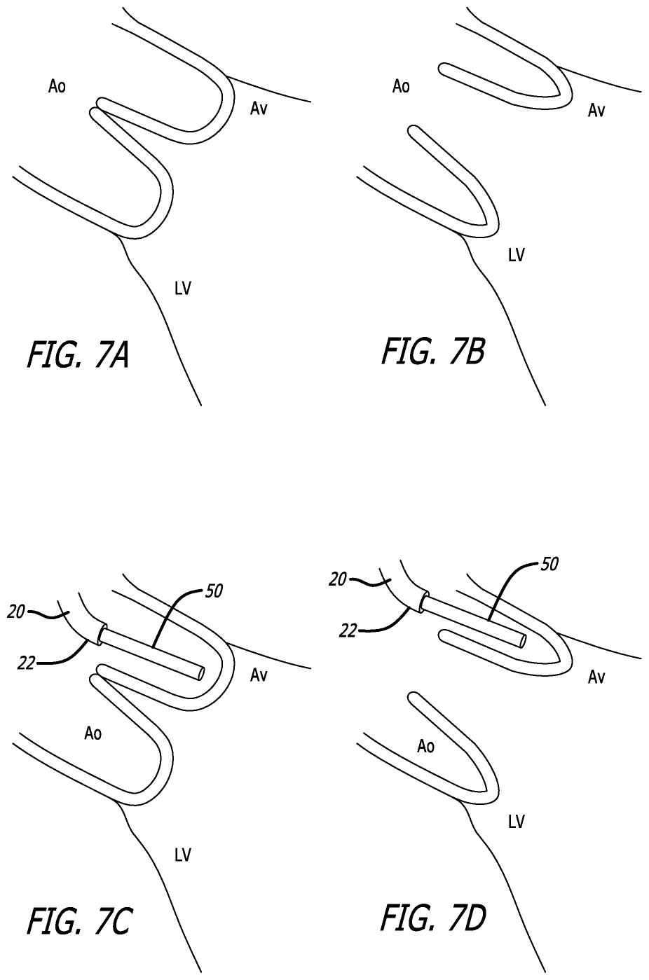

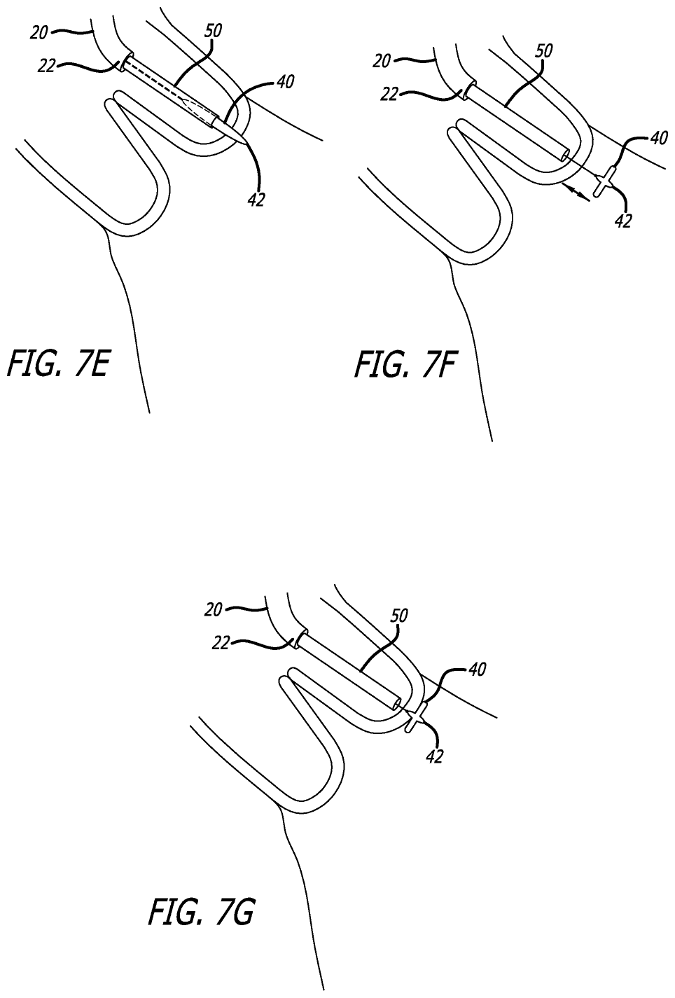

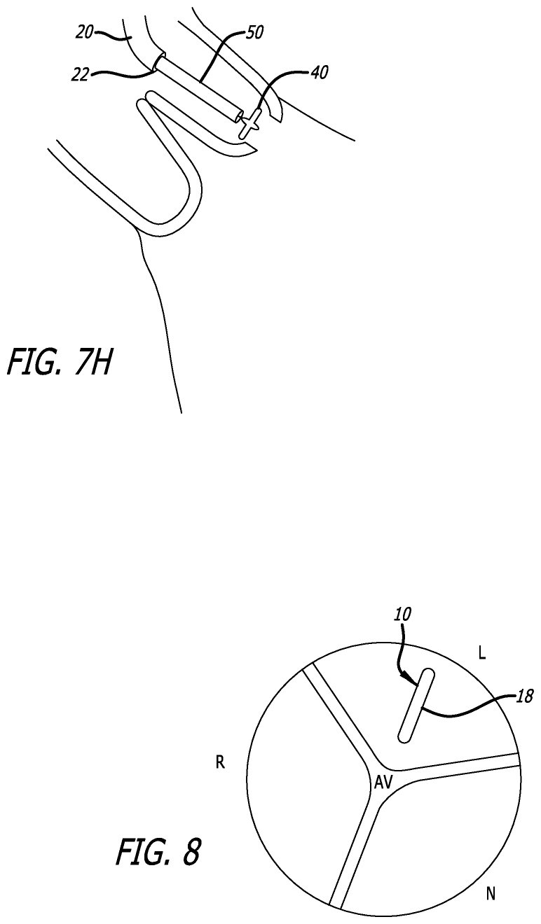

Referring now to FIGS. 7A-7H an embodiment of a method of the invention is described in which the devices of the invention are used to make a modification to an aortic valve AV. By way of reference, FIG. 7A shows the aortic valve AV connecting the left ventricle LV to the ascending aorta Ao, closed during diastole. FIG. 7B, also by way of reference, shows the aortic valve AV open during systole.

Beginning with FIG. 7C, an embodiment of a method of the invention begins by navigating a steerable guide catheter 20 to a retro-aortic position through the ascending aorta and directing it toward a targeted leaflet, and advancing a delivery catheter through the distal end 22 of the guide catheter 20. FIG. 7C shows the delivery catheter 50 being advanced to the left aortic cusp during diastole, and FIG. 7D shows the delivery catheter 50 maintaining its position during systole. The system of the invention may be used to place the delivery catheter 50 in any one of the coronary cusps (i.e. left, right, or non-coronary).

In FIG. 7E, a cutting element 40 is advanced from the delivery catheter such that a sharpened distal crossing portion 42 pierces the leaflet and passes through. It may be advantageous to advance the delivery catheter 50 until it abuts the leaflet to prevent buckling of the cutting element 40 and facilitating more accurate placement thereof. In at least some embodiments, the cutting element 40 is an ablative electrical element and may be energized in order to pass through the leaflet. Other modalities of cutting are known to those of skill in the art, including but not limited to, mechanical cutting, cryoablative cutting, laser cutting, RF cutting, ultrasonic cutting, electrosurgical cutting, etc. In other embodiments, an additional piercing wire may be advanced just distal of, and in unison with the cutting element 40 in order effect passing the cutting element 40 through the leaflet. FIG. 7F shows the cutting element 40 having passed through the leaflet and expanded to deployed configuration for creating a modification of a desired shape.

In FIG. 7G, the cutting element 40 is retracted, while energized if applicable, through the leaflet in the expanded configuration such that it begins forming the modification. In FIG. 7H, the cutting element 40 has passed through the leaflet and the modification has been formed.

FIG. 8 shows a completed view of the modification 10 made to the aortic valve leaflet using the method of FIGS. 7A-7H. The modification 10 consists of a single slit 18 formed in the middle of the leaflet, separated from the coapting edges.

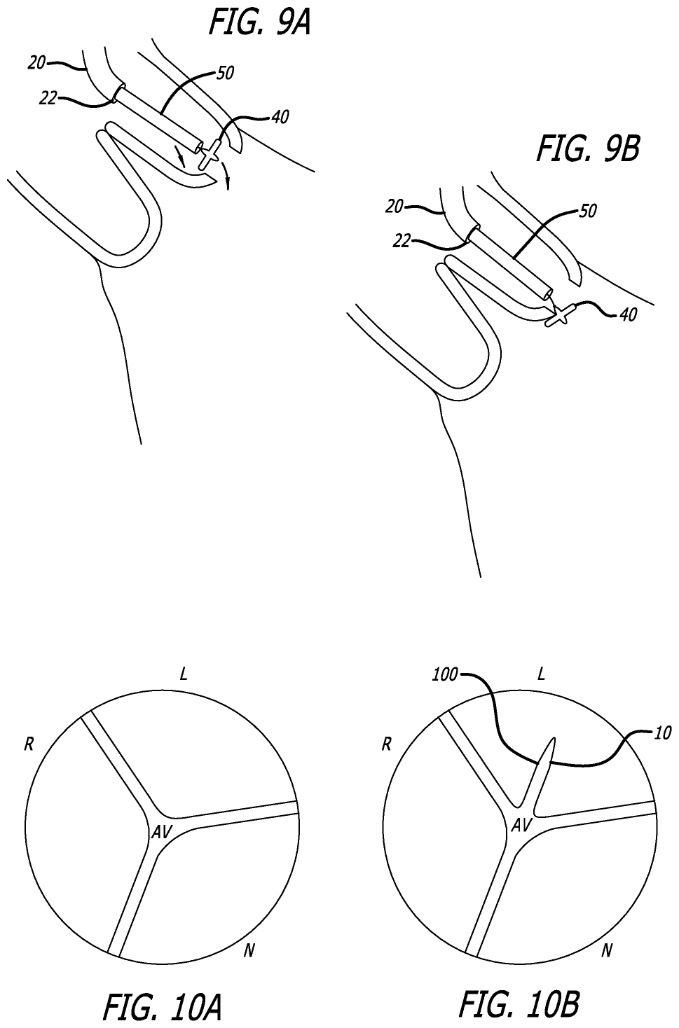

Referring now to FIGS. 9A-9B, additional steps of the method are shown, which may be used to create additional modification shapes, if desired, using the same cutting element 40. In FIG. 9A, the steerable guide catheter 20 and the delivery catheter 50 are repositioned by rotation, flexion, or extension to a different location on the leaflet with the cutting element 40 retracted from the leaflet and then advanced past the leaflet as described above or using the slit already formed. The cutting element 40 is then retracted through the leaflet to form the additional modification.

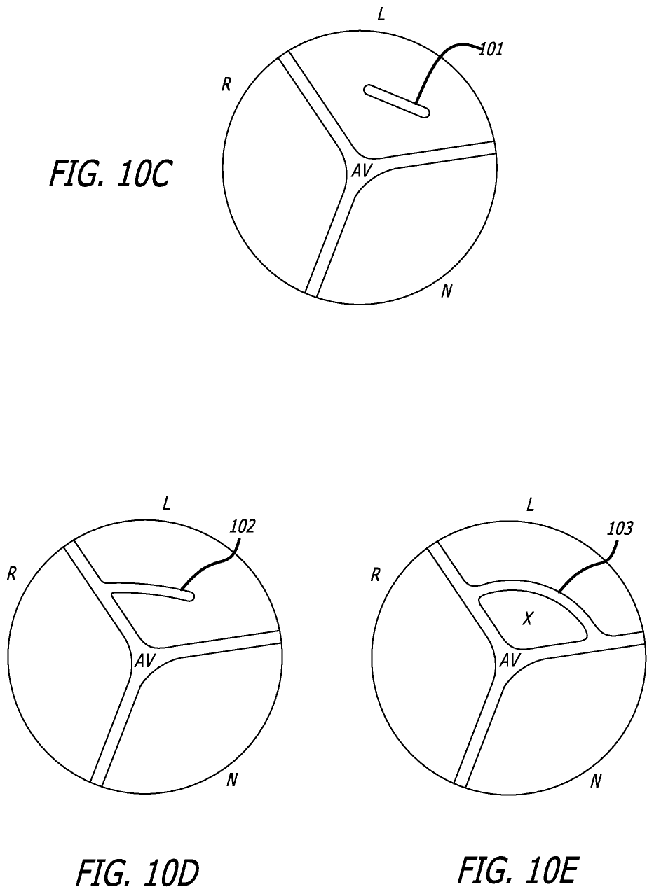

Referring to FIGS. 10A-10F, there are shown non-limiting examples of modifications 10 made to the aortic valve using the aforementioned method. FIG. 10A is a reference diagram of an unmodified aortic valve AV. FIG. 10B shows a modification 10 in the form of a slit 100 that extends from the center of a leaflet radially to a point in the middle of the leaflet. FIG. 10C shows a slit 101 formed in a center of a leaflet, away from the edges, but oriented roughly parallel to the aortic valve circumference. FIG. 10D shows a slit 102 formed from a leaflet edge and running roughly parallel to the aortic valve circumference to a point within the leaflet. FIG. 10E shows a slit 103 extending from one free edge to another free edge, effectively leading to the ability to remove the aortic valve leaflet tissue portion X.

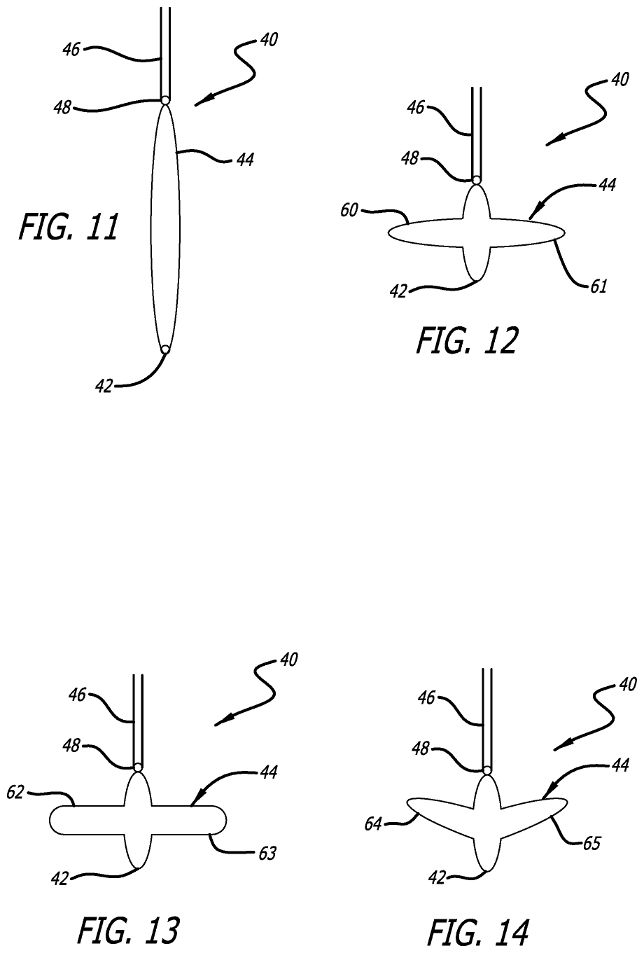

With regard to the various embodiments of the cutting element 40 of the invention, attention is directed first to FIG. 11, which shows that the cutting element 40 generally includes a cutting unit 44 that has a distal crossing portion 42, a push rod 46 used to advance the cutting element 40 through the delivery catheter 50, and a connecting element 48 that connects the cutting unit 44 to the push rod 46.

The cutting unit 44 is generally a looped wire having a first configuration and a second, deployed configuration. The second configuration is, in at least one embodiment, heat set into the wire such that the second configuration is assumed upon release from the delivery catheter. In at least one embodiment, the wire is formed from a memory metal such as Nitinol. In one embodiment, the cutting unit 44 is self-expanding with a shape that allows engagement of a leaflet from one or both sides of the valve. In another embodiment, the cutting unit 44 has exposed elements for electrification and cutting of a leaflet. In yet another embodiment, the cutting unit 44 has sharp edges to mechanically cut the leaflet. In yet another embodiment, the cutting unit 44 can be passed through the leaflet, and then expanded or electrified to facilitate modification.

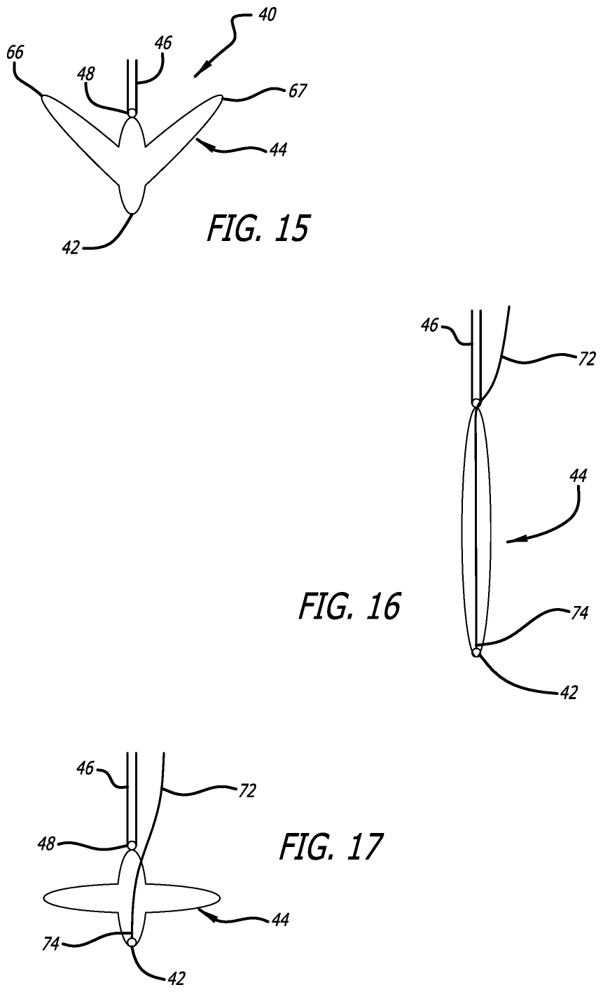

The cutting unit 44 can be modified with various shapes to suit the anatomy of the heart valve and planned subsequent therapy. FIGS. 12-15 show embodiments of cutting elements 40 that differ only in the shapes of the cutting units 44. FIG. 12 shows a cutting unit 44 in a second, expanded configuration in which oblong, elliptical wings 60 and 61 are formed. FIG. 13 shows a cutting unit 44 in a second, expanded configuration in which oblong, oval wings 62 and 63 are formed. FIG. 14 shows a cutting unit 44 in a second, expanded configuration in which swept-back wings 64 and 65 are formed. FIG. 15 shows a cutting unit 44 in a second, expanded configuration in which elongate swept-back wings 66 and 67 are formed.

FIGS. 16 and 17 show an embodiment of a cutting element 70 that utilizes a tether 72 in order to manually change the configuration of the cutting unit 44 from the first configuration to the second configuration. Cutting element 70, like the other embodiments, includes a includes a cutting unit 44 that has a distal crossing portion 42, a push rod 46 used to advance the cutting element 40 through the delivery catheter 50, and a connecting element 48 that connects the cutting unit 44 to the push rod 46. The tether 72 runs from a proximal end of the catheter, where it is attached to a mechanism that allows a user to pull on the tether. A distal end 74 of the tether 72 is attached to the distal crossing portion 42 of the cutting unit 44. Pulling on the tether 72 retracts the distal crossing portion 42, causing the cutting unit 44 to assume the second configuration, as seen in FIG. 17.

As used herein, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an ingredient or element may still actually contain such item as long as there is generally no measurable effect thereof.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description should be read to include one or at least one and the singular also includes the plural unless it is obvious that it is meant otherwise.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A valve leaflet modification device comprising:
a steerable guide catheter;
a piercing element extendable from a distal end of the steerable guide catheter and having tissue engagement features;
a cutting element extendable from the distal end of the steerable guide catheter and expandable from a first configuration to a second configuration;
wherein said cutting element and said piercing element are translatable relative to each other to trap a valve leaflet between the cutting element and the piercing element, allowing the cutting element to modify the leaflet.

2. The device of claim 1 wherein the cutting element comprises an electrical cutting unit.

3. The device of claim 1 wherein the cutting element comprises a mechanical cutting unit.

4. The device of claim 1 wherein the tissue engagement features comprise barbs.

5. The device of claim 1 wherein the cutting element self-expands from said first configuration to said second configuration.

6. The device of claim 1 wherein the cutting element comprises a wire.

7. The device of claim 1 wherein the cutting element comprises a cutting unit, a push rod, and a connecting element connecting the cutting unit to the push rod.

8. A system for making modifications to native leaflets comprising:
a steerable guide catheter;
a delivery catheter extendable from a distal end of the steerable guide catheter;
a cutting element extendable from the distal end of the steerable guide catheter and expandable from a first configuration to a second configuration, the cutting element including:
a cutting unit having a distal crossing portion;
a push rod; and,
a connecting element that connects the cutting unit to the push rod.

9. The system of claim 1 wherein the system further comprises a tether extending from a proximal end of the system, through the delivery catheter and connected to the distal crossing portion and usable to expand the cutting element to the second configuration.

10. The system of claim 8 wherein the cutting element comprises an electrical cutting unit.

11. The system of claim 8 wherein the cutting element comprises a mechanical cutting unit.

12. The system of claim 8 wherein the tissue engagement features comprise barbs.

13. The system of claim 8 wherein the cutting element self-expands from said first configuration to said second configuration.

14. The system of claim 8 wherein the cutting element comprises a wire.

15. A method for treating a valve leaflet comprising:
advancing a cutting element through a catheter to a targeted valve leaflet;
expanding the cutting element from a first configuration to a second configuration;
engaging the valve leaflet with the cutting element; and,
modifying the valve leaflet with the cutting element.

16. The method of claim 15 wherein expanding the cutting element from a first configuration to a second configuration comprises releasing the cutting element from a catheter such that the cutting element self-expands to the second configuration.

17. The method of claim 15 wherein engaging the valve leaflet with the cutting element comprises advancing the cutting element distally while retracting the leaflet proximally with a piercing element.

18. The method of claim 15 wherein engaging the valve leaflet with the cutting element comprises advancing the cutting element distally through the leaflet in the first configuration and retracting the cutting element proximally through the leaflet in the second configuration.

19. The method of claim 15 wherein modifying the valve leaflet with the cutting element comprises forming at least one slit in the leaflet.

20. The method of claim 19 wherein forming at least one slit in the leaflet comprises forming a slit in a middle of the leaflet spaced apart from the edges.
